(19) United States
(12) Patent Application Publication
(10) Pub. No.: US 2004/0260225 A1
(43) Pub. Date: Dec. 23, 2004

(54) Use of a wound dressing in treatment of pressure sores

(76) Inventors: Steven M. Bishop, Deeside (GB); Bryan Griffiths, Upton (GB); Helen L. Shaw, Widnes (GB); Simon M. Adams, Holywell (GB)

Correspondence Address: BRISTOL-MYERS SQUIBB COMPANY, 100 HEADQUARTERS PARK DRIVE, SKILLMAN, NJ, 08558, US

(21) Appl. No.: 10/841,046

(22) Filed: May 7, 2004

(30) Foreign Application Priority Data
May 9, 2003 (GB) 0310721.6

(51) Int. Cl. A61F 13/00

(52) U.S. Cl. 602/54

(57) ABSTRACT

The use of an absorbent layer comprising gel forming fibres in the manufacture of a wound dressing which forms a gel cushioning structure on the absorption of exudate from a wound for use in the treatment of pressure sores.

This invention relates to the use of a wound dressing in the treatment of pressure sores and particularly pressure sores on the heel of the foot.

Pressure sores are sometimes known as bedsores, decubitus ulcers or trophic ulcers and arise in tissues over the sacrum, ischia, greater trochanters, and heels. Pressure sores are characterised by ischemic necrosis and ulceration of tissues overlying a bony prominence that has been subjected to prolonged pressure against an external object e.g. a bed, wheelchair, cast or splint. Heel sores are known to be particularly common in certain patient groups such as the elderly, orthopaedic patients and diabetic patients.

In the past those involved in the treatment of these patient groups have tried to prevent the formation of pressure sores or treat such sores by the use of pressure relieving devices and mattresses. Little attention has been paid however to the pressure relief that may be obtained by the choice of wound dressing on the treatment of pressure sores. That is, the choice of wound dressing has been dictated solely by the wound not that it is a wound that has arisen as a result of pressure.

Surprisingly we have found that certain wound dressings known for use in the treatment of wounds can be particularly effective in the treatment of pressure sores.

Accordingly the invention provides the use of an absorbent layer comprising gel forming fibres in the manufacture of a wound dressing which forms a gel cushioning structure on the absorption of exudate from the wound for use in the treatment of pressure sores.

In particular we have found that absorbent layers which are able to absorb and retain absorbed exudate under pressure are preferred for use in the present invention. In particular fibres for use in the present invention are preferably hygroscopic fibres which upon the uptake of wound exudate become moist and slippery and eventually form a gel. The absorbent layer may comprise a fibrous layer of gel forming fibres of alginate, viscose, modified cellulose, cellulose, polyester, polypropylene and co-polymers thereof, pectin, chitosan fibres, hyaluronic acid fibres or other polysaccharide fibres or fibres derived from gums. Preferably the gel forming fibres for use in the present invention have an absorbency of at least 15 g/g of water as measured by the 1996 British Pharmacopoeia free swell test, more preferably between 25 g/g and 60 g/g. Most preferred are highly absorbent gel forming fibres such as modified cellulose fibres as described in WO93/12275 or WO94/16746 both to Akzo Nobel or WO94/17227 to E. R. Squibb and Sons. By highly absorbent with respect to the fibre it is meant that the fibre can absorb at least 25 g/g of deionised water as measured by the 1996 British Pharmacopoeia free swell test.

The gel forming fibres used in the absorbent layer may be mixed or blended to form a composite layer or may be fibres made of a mixture of any of the above ingredients.

The wound dressing may be adhesive or non-adhesive.

In an adhesive variant the wound dressing may include an adhesive layer. The adhesive layer may extend over the absorbent layer and may form the wound contact surface of the wound dressing. Alternatively, or additionally, the adhesive layer may form an adhesive edge around the absorbent layer. The absorbent layer may form an ‘island’ on the adhesive layer.

The adhesive layer of the dressing may adhere the dressing to the skin. Preferably, if the adhesive layer forms the wound contact surface, the adhesive layer is apertured. In use, the apertures allow the absorbent layer to form a quilted gel cushioning structure when exudate is absorbed.

We have found that an adhesive layer which itself is able to absorb exudate and swell while maintaining adherence of the dressing to the wound is most useful. Preferably the adhesive composition comprises a homogeneous blend of one or more water soluble hydrocolloids and one or more low molecular weight polyisobutylenes such as are described in EP-B-92999 incorporated herein by reference. The water soluble hydrocolloids may be selected from sodium carboxymethylcellulose, pectin, gelatin, guar gum, locust bean gum, gum karaya and mixtures thereof. The polyisobutylenes may be selected from low molecular weight polyisobutylenes having a viscosity average molecular weight of from 36,000 to 58,000 Florey.

Alternatively the adhesive composition may comprise a homogeneous blend of one or more hydrocolloids, one or more low molecular weight polyisobutylenes one or more styrene block copolymers, mineral oil, butyl rubber, a tackifier and small amounts of optional components. By selection of specific ranges of the amounts of the above listed components, adhesive compositions may be prepared having good adhesion to the skin and stretchability. Such compositions and the preparation thereof are disclosed in EP-B-130061 incorporated herein by reference.

We have observed that when a wound dressing comprising an absorbent layer which swells on absorption of exudate to form a gel cushioning structure is used the effects of pressure on a wound are alleviated by the action of exudate on the dressing.

This has the advantage that pressure sores when treated according to the use or method of the present invention experience a relief from the pressure being applied to them which aids healing.

Preferred embodiments of the present invention will now be described by way of example with reference to the accompanying drawings in which:

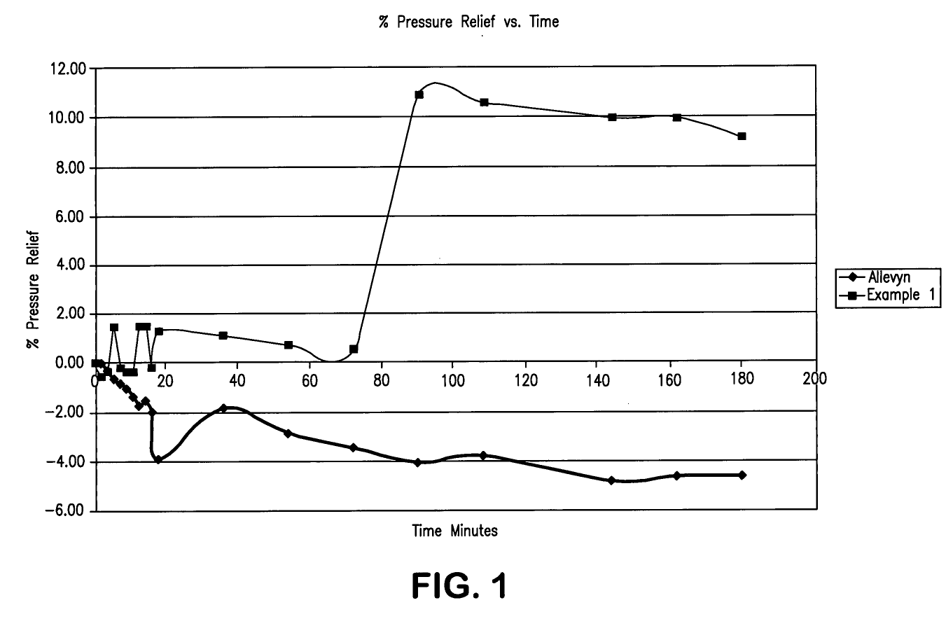

FIG. 1 is a graph of pressure relief versus time measured in a wound model as described in Example 1.

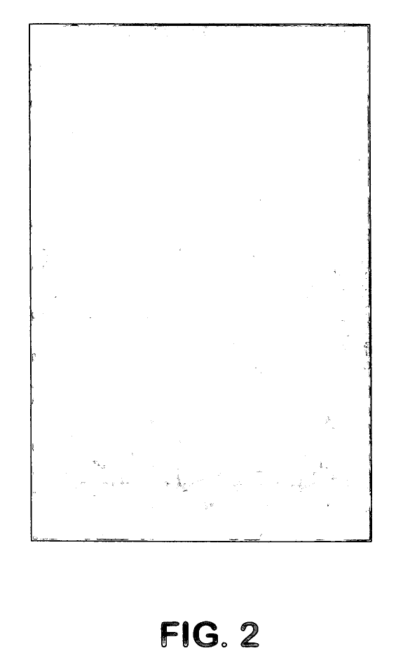

FIG. 2 is a view of the wound contact layer of the wound dressing after absorption of exudate.

Preferred embodiments of the invention will now be illustrated in the following examples:

### EXAMPLE 1

A multi layered dressing was made by blending textile and gel-forming fibres such as those described in WO93/12275 and sold as a fibrous dressing in the product AQUACEL™ ex ConvaTec in a 50/50 blend via mixing through a pre-opener and carding machine. The blended fibres were then cross-folded to the correct density, approximately 100 g/m², and needle-punched to provide an appropriate tensile strength, at least 4N/cm for the final non-woven absorbent layer. The absorbent layer was combined with a spread layer comprising viscose/polyester net with a high lateral wicking capability such as OCD ex BSF non-wovens and a transmission layer comprising a polyurethane foam/film laminate. The layers were arranged such that the spread layer was positioned between the absorbent layer and the transmission layer and the three layers were formed into a dressing by lamination under application of heat and pressure. Such a dressing is described in WO 00/41661 to Bristol-Myers Squibb Company.

The dressing also included an adhesive layer overlying the absorbent layer. The adhesive layer formed the wound contact surface and was also provided with perforations.

The dressing was placed in a device intended to simulate a pressure sore on the heel. The device comprised a pressure mat having an array of pressure sensors which conveyed readings of point pressure to a computer equipped with analysing software and a prosthetic foot touching the mat and in an orientation that would be expected of a bed ridden patient. The foot was loaded by the use of ankle weights to simulate the forces experienced by an actual foot in that situation. A wound dressing was applied to the heel of the foot and sodium calcium chloride solution was pumped into the dressing using syringe pumps to simulate typical exudation and rate from a wound. The pressure mat and software recorded the changes in pressure distribution over the three hours of the test. The results were calculated as a percentage pressure relief from the initial pressure measurement prior to the solution being pumped into the dressing corrected for the pressure map of just the foot alone.

The results are shown graphically in FIG. 1 which shows that as a consequence of the cushioning effect experienced by the heel as the dressing absorbs exudate there was some redistribution of pressure and relief.

### EXAMPLE 2

Also presented in FIG. 1 are the results obtained by subjecting a commercial foam dressing to the same measurement device and regime. The commercial foam dressing is Allevyn ex Smith and Nephew which is a dressing comprising a non-adherent wound contact layer, a soft and absorbent foam layer and an outer film layer. The main differences between this dressing and that used in the present invention are that the Allevyn dressing comprises a foam as its absorbent layer which although is absorbent, does not retain exudate under pressure and that the wound contact layer is non-adhesive.

From FIG. 1 it can be seen that the % pressure relief experienced by the foot having a dressing comprising an absorbent and retentive absorbent layer increases rapidly after exposure of the dressing to a simulated wound for 70 minutes. The foam dressing however exhibits no pressure relief.

These results suggest that a health care professional when selecting a dressing for the treatment of a pressure sore should select a dressing which gives the cushioning effect in use of the present invention.

### EXAMPLE 3

FIG. 2 shows the wound contact surface of the dressing of Example 1 for use in the present invention after the absorption of exudate. The quilting effect caused by the absorption of exudate and consequent swelling of the absorbent and apertured adhesive layers is clearly visible.

1) A method for preparing a wound dressing for use in the treatment of pressure sores comprising including in said wound dressing gel forming fibres which form a gel cushioning structure on the absorption of exudate from said pressure sores.

2) The method as claimed in claim 1 wherein the absorbent layer is able to absorb and retain wound exudate under pressure.

3) The method as claimed in claim 1 wherein the absorbent layer comprises gel forming fibres of alginate, viscose, modified cellulose, cellulose, polyester, polypropylene and co-polymers thereof, pectin, chitosan fibres, hyaluronic acid fibres or other polysaccharide fibres, fibres derived from gums, or mixtures thereof.

4) The method as claimed in claim 1 wherein the wound dressing includes an adhesive layer.

5) The method as claimed in claim 4 wherein the adhesive layer overlies the absorbent layer.

6) The method as claimed in claim 4 wherein the adhesive layer forms an adhesive edge around the absorbent layer.

7) The method as claimed in claim 5 wherein the adhesive layer forms the wound contact surface of the wound dressing.

8) The method as claimed in claim 7 wherein the adhesive layer is apertured.

9) The method as claimed in claim 8 wherein the wound dressing forms a quilted gel cushioning structure when exudate is absorbed.

10) The method as claimed in claim 5 wherein the adhesive layer is able to absorb exudate and swell while maintaining adherence of the dressing to the wound.

11) The method as claimed in claim 5 wherein the adhesive layer comprises a homogeneous blend of one or more water soluble hydrocolloids and one or more low molecular weight polyisobutylenes.

12) A method of treating a pressure sore comprising applying a wound dressing with an absorbent layer comprising gel forming fibres to the pressure sore, said gel forming fibres being arranged to absorb exudate from the pressure sore to form a gel cushioning structure.

13) The method as claimed in claim 12 wherein the absorbent layer is able to absorb and retain wound or pressure sore exudate under pressure.

14) The method as claimed in claim 12 or wherein the absorbent layer comprises gel forming fibres of alginate, viscose, modified cellulose, cellulose, polyester, polypropylene and co-polymers thereof, pectin, chitosan fibres, hyaluronic acid fibres or other polysaccharide fibres, fibres derived from gums, or mixtures thereof.

15) The method as claimed in claim 12 wherein the wound dressing includes an adhesive layer.

16) The method as claimed in claim 15 wherein the adhesive layer overlies the absorbent layer.

17) The method as claimed in claim 15 wherein the adhesive layer forms an adhesive edge around the absorbent layer.

18) The method as claimed in claim 16 wherein the adhesive layer forms the wound contact surface of the wound dressing.

19) The method as claimed in claim 18 wherein the adhesive layer is apertured.

20) The method as claimed in claim 19 wherein the wound dressing forms a quilted gel cushioning structure when exudate is absorbed.

21) The method as claimed in claim 15 wherein the adhesive layer is able to absorb exudate and swell while maintaining adherence of the dressing to the wound.

22) The method as claimed in claim 15 wherein the adhesive layer comprises a homogeneous blend of one or more water soluble hydrocolloids and one or more low molecular weight polyisobutylenes.
